(19) United States
(12) Patent Application Publication
(10) Pub. No.: US 2003/0040534 A1
(43) Pub. Date: Feb. 27, 2003

(54) Enantiomers of N-[[2'-[[(4,5-dimethyl-3-isoxazolyl)amino]sulfonyl]-4-(2-oxazolyl)[1,1'-biphenyl]-2-yl]methyl]-N,3,3-trimethylbutanamide

(76) Inventors: David E. Hughes, Pennington, NJ (US); Beth C. Seidenberg, Basking Ridge, NJ (US)

Correspondence Address: Stephen B. Davis, Bristol-Myers Squibb Company, Patent Department, P.O. Box 4000, Princeton, NJ, 08543-4000, US

(21) Appl. No.: 10/121,520

(22) Filed: Apr. 12, 2002

Related U.S. Application Data
(60) Provisional application No. 60/284,080, filed on Apr. 16, 2001.

(51) Int. Cl. A61K 31/422; C07D 413/02

(52) U.S. Cl. 514/374; 548/235

(57) ABSTRACT

Endothelin antagoninst N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide surprisingly exists as separable enantiomeric atropisomers. The (+) dextrorotatory atropisomer demonstrates remarkably higher potency than either the (−) levorotatory atropisomer or the racemate.

### RELATED APPLICATIONS

This application claims benefit to provisional application U.S. Serial No. 60/284,080 filed Apr. 16, 2001. The entire teachings of the referenced applications are incorporated herein by reference.

### FIELD OF THE INVENTION

The present invention relates to enantiomers of N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide, which is a potent endothelin antagonist.

### BRIEF DESCRIPTION OF THE INVENTION

The present invention provides for enantiomers of N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide. Specifically, the present invention provides for enantiomeric atropisomers of N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide.

As described in U.S. Pat. No. 6,043,265, N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide is a potent endothelin antagonist having excellent oral bioavailability, duration of action and pre-systemic metabolic stability within the gastrointestinal tract, and is thus particularly useful in the treatment of endothelin-related disorders. Until our discovery, it had been believed that N-[[2′-[[(4,5- dimethyl-3-isoxazolyl)amino]sulfonyl]-4-(2-oxazolyl)[1,1′-biphenyl]-2-yl]methyl]-N,3,3-trimethylbutanamide existed as a rapidly interconverting racemic mixture of atropisomeric enantiomers. We have discovered that N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide surprisingly exists as isolatable enantiomeric atropisomers, and have further discovered that the (+) dextrorotatory atropisomer demonstrates remarkably higher potency than either the (−) levorotatory atropisomer or the racemate. The present invention thus provides for the (+) dextrorotatory atropisomer of N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide, as well as pharmaceutical compositions comprising this (+) dextrorotatory atropisomer, and methods of treating endothelin-related disorders comprising the administration therapeutically effective amount of the (+) dextrorotatory atropisomer to a patient in need of such treatment.

### DETAILED DESCRIPTION OF THE INVENTION

The compound N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide has the following structure:

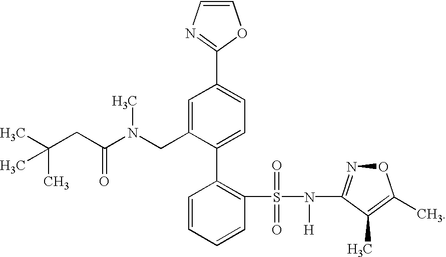

This substituted biphenyl compound possesses a stereogenic or chiral axis of rotation as depicted in the following formula:

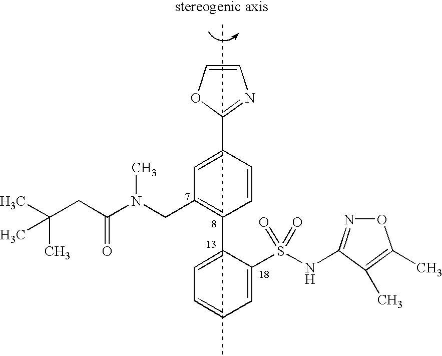

While the existence of this stereogenic axis of rotation would be apparent from an inspection of the chemical structure, one of ordinary skill would not expect N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide to exist as resolvable atropisomers. In the case of biphenyl compounds, generally only compounds that are substituted at each of the 2, 2′, 6, and 6′ positions possess rotational barriers of high enough energy to allow the resolution of enantiomeric atropisomers. See, e.g., Lowry, T. H., and Schueller Richardson, K. Mechanism and Theory in Organic Chemistry pp. 122-125 (1981) (2d ed.). Thus, one would expect N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide to exist as a rapidly interconverting racemic mixture of atropisomeric enantiomers.

Contrary to this expectation however, we have discovered that N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide can be readily separated into its enantiomeric atropisomers, and we have further discovered that the (+) dextrorotatory atropisomer demonstrates remarkably higher potency than either the (−) levorotatory atropisomer or the racemate.

Any and all salts of (+) dextrorotatory atropisomer of N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide are contemplated herein, and in particular those formed with inorganic or organic bases. Pharmaceutically acceptable (i.e., non-toxic, physiologically acceptable) salts are preferred, although other salts are also useful, for example, in isolation or purification of the present compounds.

Preferred are alkali metal salts such as sodium, potassium and lithium salts, alkaline earth metal salts such as calcium and magnesium salts, as well as salts formed with organic bases (e.g., organic amines) such as dicyclohexylamine, t-butyl amine, benzathine, N-methyl-D-glucamide and hydrabamine, and with amino acids such as arginine, lysine and the like.

The compounds of the present invention are antagonists of ET-1, ET-2 and/or ET-3 and are useful in treatment of conditions associated with increased ET levels (e.g., dialysis, trauma and surgery) and of all endothelin-dependent disorders. They are thus useful as antihypertensive agents. By the administration of a composition having one (or a combination) of the compounds of this invention, the blood pressure of a hypertensive mammalian (e.g., human) host is reduced. They are also useful in portal hypertension, hypertension secondary to treatment with erythropoietin and low renin hypertension.

The compounds of the present invention are also useful in the treatment of disorders related to renal, glomerular and mesangial cell function, including acute (such as ischemic, nephrotoxic, or glomerulonephritis) and chronic (such as diabetic, hypertensive or immune-mediated) renal failure, diabetic nephropathy, glomerular injury, renal damage secondary to old age or related to dialysis, nephrosclerosis (especially hypertensive nephrosclerosis), nephrotoxicity (including nephrotoxicity related to imaging and contrast agents and to cyclosporine), renal ischemia, primary vesicoureteral reflux, glomerulosclerosis and the like. The compounds of this invention are also useful in the treatment of disorders related to paracrine and endocrine function. The compounds of this invention are also useful in the treatment of diabetic nephropathy, hypertension-induced nephropathy, and IGA-induced nephropathy.

The compounds of the present invention are also useful in the treatment of endotoxemia or endotoxin shock as well as hemorrhagic shock. The compounds of the present invention are also useful in alleviation of pain associated cancer, such as the pain associated with prostate cancer, and bone pain associated with bone cancer. The compounds of the present invention are further useful in the prevention and/or reduction of end-organ damage associated the cell-poliferative effects of endothelin.

The compounds of the present invention are also useful in hypoxic and ischemic disease and as anti-ischemic agents for the treatment of, for example, cardiac, renal and cerebral ischemia and reperfusion (such as that occurring following cardiopulmonary bypass surgery), coronary and cerebral vasospasm, and the like.

In addition, the compounds of this invention are also useful as anti-arrhythmic agents; anti-anginal agents; anti-fibrillatory agents; anti-asthmatic agents; anti-atherosclerotic and anti-arteriosclerotic agents (including anti-transplantation arteriosclerotic agents); additives to cardioplegic solutions for cardiopulmonary bypasses; adjuncts to thrombolytic therapy; and anti-diarrheal agents. The compounds of this invention may be useful in therapy for myocardial infarction; therapy for peripheral vascular disease (e.g., Raynaud's disease, intermittent claudication and Takayashu's disease); treatment of cardiac hypertrophy (e.g., hypertrophic cardiomyopathy); treatment of primary pulmonary hypertension (e.g., plexogenic, embolic) in adults and in the newborn and pulmonary hypertension secondary to heart failure, radiation and chemotherapeutic injury, or other trauma; treatment of central nervous system vascular disorders, such as stroke, migraine and subarachnoid hemorrhage; treatment of central nervous system behavioral disorders; treatment of gastrointestinal diseases such as ulcerative colitis, Crohn's disease, gastric mucosal damage, ulcer, inflammatory bowel disease and ischemic bowel disease; treatment of gall bladder or bile duct-based diseases such as cholangitis; treatment of pancreatitis; regulation of cell growth; treatment of benign prostatic hypertrophy; restenosis following angioplasty or following any procedure including transplantation and stenting; therapy for congestive heart failure including inhibition of fibrosis; inhibition of left ventricular dilatation, remodeling and dysfunction; and treatment of hepatotoxicity and sudden death. The compounds of this invention are useful in the treatment of sickle cell disease including the initiation and/or evolution of the pain crises of this disease; treatment of the deleterious consequences of ET-producing tumors such as hypertension resulting from hemangiopericytoma; treatment of early and advanced liver disease and injury including attendant complications (e.g., hepatotoxicity, fibrosis and cirrhosis); treatment of spastic diseases of the urinary tract and/or bladder; treatment of hepatorenal syndrome; treatment of immunological diseases involving vasculitis such as lupus, systemic sclerosis, mixed cryoglobulinemia; and treatment of fibrosis associated with renal dysfunction and hepatotoxicity. The compounds of this invention are useful in therapy for metabolic and neurological disorders; cancer; insulin-dependent and non insulin-dependent diabetes mellitus; neuropathy; retinopathy; epilepsy; hemorrhagic and ischemic stroke; bone remodeling; psoriasis; and chronic inflammatory diseases such as arthritis, rheumatoid arthritis, osteoarthritis, sarcoidosis and eczematous dermatitis (all types of dermatitis).

The compounds of this invention are additionally useful in the treatment of disorders involving bronchoconstriction and disorders of chronic or acute pulmonary inflammation such as chronic obstructive pulmonary disease (COPD) and adult respiratory distress syndrome (ARDS).

The compounds of this invention are also useful in the treatment of sexual dysfunction in both men (erectile dysfunction, for example, due to diabetes mellitus, spinal cord injury, radical prostatectomy, psychogenic etiology or any other cause) and women by improving blood flow to the genitalia, especially, the corpus cavernosum.

The compounds of this invention are also useful in the treatment of dementia, including Alzheimer's dementia, senile dementia and vascular dementia.

The compounds of the present invention may be employed alone or in combination with other suitable therapeutic agents useful in the treatment of endothelin-dependent disorders or other related disorders. For example, the compounds of this invention can be formulated in combination with endothelin converting enzyme (ECE) inhibitors, such as phosphoramidon; thromboxane receptor antagonists such as ifetroban; potassium channel openers; thrombin inhibitors (e.g., hirudin and the like); growth factor inhibitors such as modulators of PDGF activity; platelet activating factor (PAF) antagonists; anti-platelet agents such as GPIIb/IIIa blockers (e.g., abciximab, eptifibatide, and tirofiban), P2Y(AC) antagonists (e.g., clopidogrel, ticlopidine and CS-747), and aspirin; anticoagulants such as warfarin, low molecular weight heparins such as enoxaparin, Factor VIIa inhibitors, and Factor Xa inhibitors such as those described in U.S. Ser. No. 09/496,571 filed Feb. 2, 2000 (attorney docket HA 723); renin inhibitors; angiotensin converting enzyme (ACE) inhibitors such as captopril, zofenopril, fosinopril, ceranapril, alacepril, enalapril, delapril, pentopril, quinapril, ramipril, lisinopril and salts of such compounds; neutral endopeptidase (NEP) inhibitors; vasopepsidase inhibitors (dual NEP-ACE inhibitors) such as omapatrilat and gemopatrilat; HMG CoA reductase inhibitors such as pravastatin, lovastatin, atorvastatin, simvastatin, NK-104 (a.k.a. itavastatin, or nisvastatin or nisbastatin) and ZD-4522 (a.k.a. rosuvastatin, or atavastatin or visastatin); squalene synthetase inhibitors; fibrates; bile acid sequestrants such as questran; niacin; anti-atherosclerotic agents such as ACAT inhibitors; MTP inhibitors such as those described in U.S. Ser. No. 09/007,938 filed Jan. 16, 1998 (attorney docket HX 91); calcium channel blockers such as amlodipine besylate; potassium channel activators; alpha-adrenergic agents, beta-adrenergic agents such as carvedilol and metoprolol; antiarrhythmic agents; diuretics, such as chlorothiazide, hydrochlorothiazide, flumethiazide, hydroflumethiazide, bendroflumethiazide, methylchlorothiazide, trichloromethiazide, polythiazide or benzothiazide as well as ethacrynic acid, tricrynafen, chlorthalidone, furosemide, musolimine, bumetanide, triamterene, amiloride and spironolactone and salts of such compounds; thrombolytic agents such as tissue plasminogen activator (tPA), recombinant tPA, streptokinase, urokinase, prourokinase and anisoylated plasminogen streptokinase activator complex (APSAC); anti-diabetic agents such as biguanides (e.g. metformin), glucosidase inhibitors (e.g., acarbose), insulins, meglitinides (e.g., repaglinide), sulfonylureas (e.g., glimepiride, glyburide, and glipizide), biguanide/glyburide combinations such as those described in U.S. Ser. No. 09/432,465 filed Nov. 3, 1999 (attorney docket LA 46) and U.S. Ser. No. 09/460,920 filed Dec. 14, 1999 (attorney docket LA 46a); thiozolidinediones (e.g. troglitazone, rosiglitazone and pioglitazone), and PPAR-gamma agonists; mineralocorticoid receptor antagonists such as spironolactone and eplerenone; growth hormone secretagogues such as those described in U.S. Ser. No. 09/417,180 filed Oct. 12, 1999 (attorney docket LA 25) and U.S. Ser. No. 09/506,749 filed Feb. 18, 2000 (attorney docket LA 26); aP2 inhibitors such as those described in U.S. Ser. No. 09/391,053 filed Sep. 7, 1999 (attorney docket LA 24a) and U.S. Ser. No. 09/390,275 filed Sep. 7, 1999 (attorney docket LA 24b); digitalis; ouabian; non-steroidal antiinflammatory drugs (NSAIDS) such as aspirin and ibuprofen; phosphodiesterase inhibitors such as PDE III inhibitors (e.g., cilostazol) and PDE V inhibitors (e.g., sildenafil); protein tyrosine kinase inhibitors; antiinflammatories; antiproliferatives such as methotrexate, FK506 (tacrolimus, Prograf), mycophenolate and mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel, docetaxel, and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin); cyclosporins; steroids such as prednisone or dexamethasone; gold compounds; cytotoxic drugs such as azathiprine and cyclophosphamide; TNF-alpha inhibitors such as tenidap; anti-TNF antibodies or soluble TNF receptor such as etanercept (Enbrel) rapamycin (sirolimus or Rapamune), leflunimide; and cyclooxygenase-2 (COX-2) inhibitors such as celecoxib and rofecoxib.

If formulated as a fixed dose, such combination products preferably employ the compounds of this invention within the dosage range described below and the other pharmaceutically active agent within its approved dosage range. The compounds of this invention may also be formulated with, or useful in conjunction with, antifungal and immunosuppressive agents such as amphotericin B, cyclosporins and the like to counteract the glomerular contraction and nephrotoxicity secondary to such compounds. The compounds of this invention may also be used in conjunction with hemodialysis.

The compounds of the invention can be administered in any suitable manner such as orally or parenterally to various mammalian species known to be subject to such maladies, e.g., humans, in an effective amount such as an amount within the dosage range of about 0.1 to about 100 mg/kg, preferably about 0.2 to about 50 mg/kg and more preferably about 0.5 to about 25 mg/kg (or from about 1 to about 2500 mg, preferably from about 5 to about 2000 mg) in single or 2 to 4 divided daily doses. Effective dosage ranges for racemic N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide, are preferably from about 1.25 mg to about 20 mg per 70 kg.

The active substance can be utilized in a composition such as tablet, capsule, solution or suspension containing, e.g., about 5 to about 500 mg per unit dosage of a compound or mixture of compounds of the present invention or in topical form for wound healing (such as 0.01 to 5% by weight compound of the invention, 1 to 5 treatments per day). The present compounds may be compounded in a conventional manner with a physiologically acceptable vehicle or carrier, excipient, binder, preservative, stabilizer, flavor, etc., or with a topical carrier such as Plastibase (mineral oil gelled with polyethylene) as called for by accepted pharmaceutical practice.

The compounds of the invention may also be administered topically to treat peripheral vascular diseases and as such may be formulated as a cream or ointment.

The compounds of the present invention can also be formulated in compositions such as sterile solutions or suspensions for parenteral administration. For example, about 0.1 to 500 milligrams of a compound of the invention may be compounded with a physiologically acceptable vehicle, carrier, excipient, binder, preservative, stabilizer, etc., in a unit dosage form as called for by accepted pharmaceutical practice. The amount of active substance in these compositions or preparations is preferably such that a suitable dosage in the range indicated is obtained.

The present invention thus provides novel methods of using, and pharmaceutical compositions containing, the novel compounds described herein. The present invention especially contemplates methods of treating endothelin-related disorders in a mammal, which comprise administering to a mammal an effective endothelin-related disorder treating amount of a compound of the present invention. The present invention also especially contemplates pharmaceutical compositions for the treatment of endothelin-related disorders, comprising a compound of the present invention in an amount effective therefor and a physiologically acceptable vehicle or carrier. A compound of the invention may, for example, be employed in the present methods or pharmaceutical compositions alone, in combination with one or more other compounds of the invention and/or in combination with at least one other active agent such as an angiotensin II (AII) receptor antagonist, renin inhibitor, angiotensin converting enzyme (ACE) inhibitor, dual neutral endopeptidase (NEP)-ACE inhibitor, diuretic, or cardiac glycoside, or other active agent listed above.

In the present methods, such other active agent(s) may be administered prior to, simultaneously with or following the administration of the compound(s) of the present invention. In the present pharmaceutical compositions, such other active agent(s) may be formulated with the compound(s) of the present invention, or administered separately as described above for the present methods.

Particularly preferred such methods and compositions are those for the treatment of hypertension, especially low renin hypertension (such as is described in U.S. patent application Ser. No. 60/035,825, filed Jan. 30, 1997 by J. E. Bird, entitled “Method for Preventing or Treating Low Renin Hypertension by Administering an Endothelin Antagonist” (Attorney Docket No. HA700*), incorporated herein by reference in its entirety) or pulmonary hypertension, particularly primary pulmonary hypertension; benign prostatic hypertrophy; migraine; renal, glomerular or mesangial cell disorders; endotoxemia; ischemia; atherosclerosis; restenosis; subarachnoid hemorrhage; and congestive heart failure.

The present invention will now be further described by the following working examples. These examples are meant to be illustrative rather than limiting. N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide used in the following examples was prepared according to the general procedures described in U.S. Pat. No. 5,856,507. As described in U.S. Provisional Application 60/240,902 [filed Oct. 17, 2000; attorney docket HA 762] methoxymethyl was employed in the synthesis as the nitrogen-protecting group.

### EXAMPLE 1

### Identification of Enantiomeric Atropisomers

The atropisomers were separated on a chiral phase (cellulose tris [3,5-dimethylphenylcarbamate]) liquid chromatographic column under the conditions recited in Example 2 with retention times of ca. 8 minutes for the (−) atropisomer and ca. 12 minutes for the (+) atropisomer, using photodiode array detection with a recording wavelength of 280 nm. Ultraviolet photodiode array spectra of the two species were obtained during the chromatographic run and the spectra were found to be both distinctive and superimposable, with maxima at 205.5 nm and 279.7 nm, as would be predicted for atropisomeric species. The two species were then examined by liquid chromatography with laser polarimetric detection, with the result that the earlier-eluting species was levorotatory at 670 nm and the later-eluting species was dextrorotatory at 670 nm. The species were also shown to be enantiomeric by capillary electrophoretic analysis in the presence and absence of the chiral selectors beta and gamma cyclodextrin.

The in vitro interconversion of N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide enantiomeric atropisomers was investigated using normal phase chiral HPLC with ultraviolet and laser polarimetric detection, under pseudo-physiological conditions, i.e., aqueous medium at 37° C., gastric fluid at 37° C. and human serum at 37° C. Kinetic studies indicate that the half-life of racemization in an aqueous medium at 37° C. is about 15 hours. A similar racemization half-life (15.8 hours) is observed in gastric fluid at 37° C., which leads to the conclusion that the enantiomeric interconversion is not acid catalyzed. Racemization of N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide enantiomeric atropisomers is accelerated in the presence of a human serum matrix. For example, at a concentration of 400 μg/mL in human serum at 37° C. the half-life of racemization is 2.5 hours, and at a concentration 80 μg/mL in human serum at 37° C. the half-life of racemization is 0.4 hours.

### EXAMPLE 2

### Isolation of Atropisomers

Isolation of the (+) and (−) enantiomeric atropisomers was accomplished by semi-preparative liquid chromatography using a 250×21 mm cellulose tris (3,5-dimethylphenylcarbamate) 10 μ particle stationary phase and a hexane/2-propanol/triethylamine/triflouroacetic acid (80/20.0.1/0.1% [v/v]) mobile phase in the isocratic mode. The wavelength of detection was adjusted from 254-320 nm with a photodiode array detector; the mobile phase flow rate was 20 mL/minute, the injection volume ranged from 0.5-5 mL, and the sample concentration was 20 mg/mL in 2-propanol. The atropisomers appeared at ca. 7 minutes for the (−) form and ca. 11 minutes for the (+) form. The two species were collected by use of a fraction collector and then the mobile phase in each sample was evaporated either to dryness or to a substantially lower volume. The evaporation of mobile phase was accomplished by nitrogen microprocessor-controlled evaporation or a centrifugally-assisted vacuum evaporator, both at ambient temperature. The resulting samples were then analyzed for (+) and (−) isomer purity. The purity analysis indicated that the samples contained 97% pure (−) and (+) isomer for the nitrogen evaporation and 99.2% pure (−) isomer and (+) isomer for vacuum evaporation.

The isomer purity assay procedure used a cellulose tris (3,5-dimethylphenylcarbamate) 250×4.6 mm, 5 μ particle stationary phase and a mobile phase of the same composition as was used for the semi-preparative isolation was used in the isocratic mode. The wavelength of detection was 280 nm, the column temperature was 10° C., the flow rate was 1 mL/minute, and the injection volume was 5 μL. Any sample dilution performed used 2-propanol. The isomers appeared at ca. 8 minutes for the (−) isomer and ca. 12 minutes for the (+) isomer. Quantitation was based on the computer-generated peak areas.

### EXAMPLE 3

### In vitro Binding of Atropisomers

The isolated atropisomers of Example 2 were tested for in vitro ETA binding according to the following procedure:

CHO-K1 cells expressing the human endothelin A receptor were cultured in Ham's F12 media (Gibco/BRL, Grand Island, N.Y.) with 10% fetal bovine serum (Hyclone), supplemented with 300 μg/mL Geneticin (G-418 Gibco BRL Products, Grand Island, N.Y.) and maintained at 37° C. with 5% CO₂ in a humidified incubator. Forty eight hours prior to assay, the cells were treated with 0.25% trypsin-EDTA and were seeded in Falcon, 96 well tissue culture plates at a density of 1.5×10⁴ cells/well (the monolayer should reach 80-90% confluency by the day of assay).

In the attached cell assay, culture media was aspirated from each well and the monolayers were washed with 75 μl of room temperature PBS (Mg⁺⁺, Ca⁺⁺ free). After the addition of 75 μl of cold binding buffer to each well, the plate was chilled at 4° C. for 3 minutes before the addition of competing drugs and isotope. The binding assay was performed in a total volume of 125 μl consisting of assay buffer, maintained at 4° C. (50 mM Tris, pH 7.4, including 1% BSA, and 2 μM phosphoramidon), and 25 μl of either 500 nM ET-1 (to define nonspecific binding) or competing drugs. The reaction was initiated with the addition of 25 μl of 0.25 nM [¹²⁵I]-ET-1 (New England Nuclear). Incubation was carried out with gentle orbital shaking, at 4° C., reaching equilibrium at 4 hours. The reaction was terminated by aspiration of the reaction buffer and two subsequent washes with room temperature PBS (Mg^{++, Ca}⁺⁺ free). The cells were dissociated by the addition of 100 μl of 0.5N NaOH followed by incubation for 40 minutes. Samples were then transferred from the 96 well format into tubes for counting in a Cobra gamma counter (Packard). Data was analyzed with curve fitting software by Sigma plot.

In each assay run that was performed, one sample of each atropisomer as well as one sample of racemate was tested for activity. The results of these binding assays for the 97% pure atropisomers of Example 2 are reported in Table 1. The results for the 99.2% pure atropisomers of Example 2 are reported in Table 2.

TABLE 1

| | IC₅₀ (nM) | | | |
|---|---|---|---|---|
| Enantiomer | Run 1 | Run 2 | Run 3 | Run 4 |
| Racemate | 0.12 | 0.24 | 0.21 | 0.1 |
| 97% pure (+) | 0.03 | 0.01 | 0.01 | 0.01 |
| dextrorotatory | | | | |
| 97% pure (−) | 0.41 | 0.9 | 0.6 | 1.4 |
| levorotatory | | | | |

TABLE 2

| | IC₅₀ (nM) | | | | | | | | |
|---|---|---|---|---|---|---|---|---|---|
| Enantiomer | Run 1 | Run 2 | Run 3 | Run 4 | Run 5 | Run 6 | Run 7 | Run 8 | Run 9 |
| Racemate | 0.018 | 0.08 | 0.4 | 0.3 | 0.2 | 0.4 | 0.24 | 0.16 | 0.27 |
| 99.2% pure (+) | 0.002 | 0.002 | 0.7 | 0.1 | 0.3 | 0.38 | 0.34 | 0.38 | 0.41 |
| dextrorotatory | | | | | | | | | |
| 99.2% pure (−) | 0.1 | 0.65 | 6 | 5 | 7 | 3.7 | 21 | 14 | 19 |
| levorotatory | | | | | | | | | |

We claim:

1. The compound (+) N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide or a salt thereof.

2. The compound of claim 1 which is (+) N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide or a pharmaceutically acceptable salt thereof.

3. The compound of claim 1 which is (+) N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide.

4. The compound of claim 1 which is a pharmaceutically acceptable salt of said compound, wherein said salt is a lithium, sodium, or potassium salt or a salt formed with an organic amine base.

5. A method of treating endothelin-related disorders in a mammal, which comprises administering to said mammal an effective endothelin-related disorder treating amount of a compound of claim 2.

6. A method of treating hypertension, which comprises administering an effective hypertension treating amount of a compound of claim 2.

7. A method of treating pulmonary hypertension, which comprises administering an effective pulmonary hypertension treating amount of a compound of claim 2.

8. A method of treating primary pulmonary hypertension, which comprises administering an effective primary pulmonary hypertension treating amount of a compound of claim 2.

9. A method of treating benign prostatic hypertrophy, which comprises administering an effective benign prostatic hypertrophy treating amount of a compound of claim 2.

10. A method of treating migraine, which comprises administering an effective migraine treating amount of a compound of claim 2.

11. A method of treating renal, glomerular or mesangial cell disorders, which comprises administering an effective renal, glomerular or mesangial cell disorder treating amount of a compound of claim 2.

12. A method of treating endotoxemia, which comprises administering an effective endotoxemia treating amount of a compound of claim 2.

13. A method of treating ischemia, which comprises administering an effective ischemia treating amount of a compound of claim 2.

14. A method of treating atherosclerosis, which comprises administering an effective atherosclerosis treating amount of a compound of claim 2.

15. A method of treating restenosis, which comprises administering an effective restenosis treating amount of a compound of claim 2.

16. A method of treating subarachnoid hemorrhage, which comprises administering an effective subarachnoid hemorrhage treating amount of a compound of claim 2.

17. A method of treating congestive heart failure, which comprises administering an effective congestive heart failure treating amount of a compound of claim 2.

18. The method of claim 5, wherein said compound of claim 2 is administered prior to, simultaneously with or following the administration of at least one angiotensin II (All) receptor antagonist, renin inhibitor, angiotensin converting enzyme (ACE) inhibitor, dual neutral endopeptidase (NEP)-ACE inhibitor, diuretic or cardiac glycoside.

19. A pharmaceutical composition for the treatment of an endothelin-related disorder, comprising a compound of claim 2 in an amount effective therefor and a physiologically acceptable vehicle or carrier.

20. A pharmaceutical composition of claim 19, further comprising at least one additional therapeutic agent selected from angiotensin II (All) receptor antagonists, renin inhibitors, angiotensin converting enzyme (ACE) inhibitors, vasopepsidase inhibitors, anti-platelet agents, diuretics or cardiac glycosides.

21. A pharmaceutical composition of claim 19 further comprising at least one vasopepsidase inhibitor selected from omapatrilat or gemopatrilat.

22. A pharmaceutical composition of claim 19 further comprising at least one antiplatelet agent selected from clopidigrel, ticlopidine, CS-747 or aspirin.

23. A method of treating asthma, which comprises administering an effective anti-asthmatic amount of a compound of claim 2.

24. A method of claim 11 wherein said disorder is diabetic nephropathy.

25. A method of treating intermittent claudication, which comprises administering an effective intermittent claudication treating amount of a compound of claim 2.

26. A method of treating cancer, which comprises administering an effective cancer treating amount of a compound of claim 2.
